(19) United States
(12) Patent Application Publication
(10) Pub. No.: US 2005/0051176 A1
(43) Pub. Date: Mar. 10, 2005

(54) Nasal and oral cannula apnea detection device

(76) Inventor: Michael Allen Riggins, Seattle, WA (US)

Correspondence Address: David L Garrison;Garrison & Associates, Suite 3300, 2001 Sixth Avenue, Seattle, WA, 98121-2522, US

(21) Appl. No.: 10/481,541

(22) PCT Filed: Apr. 26, 2002
(86) PCT No.: PCT/US02/13371

(30) Foreign Application Priority Data
Jun. 18, 2001 (US) 60299144

(51) Int. Cl. A61M 15/08

(52) U.S. Cl. 128/207.18

(57) ABSTRACT

A cannula (3) defined by a main body (1) having an internal passage (6). The internal passage (6) communicate with a nare (5, 7) and an oral prong or a mouthpiece (9) which is adjustable to provide a desirable signal from a patient's mouth and nose to indicate breathing by the patient.

This invention provides a novel cannula suitable for use in both nasal and oral applications, and a method of use thereof.

### BACKGROUND OF THE INVENTION

This invention relates generally to cannulas adapted for both mouth and nasal use to monitor breathing, in particular, a method of manufacturing a cannula adapted to interconnect with both nasal passages and the mouth for use to monitor breathing, especially for the detection of apnea (the absence of breathing).

Nasal cannulas are commonly used to administer gases, such as oxygen, to humans having respiratory problems. Illustrations of nasal cannulas used for this purpose are found in U.S. Pat. No. 3,802,431. Nasal cannulas have been used also for inhalation therapy, made possible by development of inhalation sensors such as described in U.S. Pat. No. 4,745,925. A nasal cannula can be used to monitor breathing and for detection of apnea when connected to an inhalation sensor.

Nasal cannulas additionally adapted to communicate with the mouth of humans to permit administration of gaseous fluids or sensing of apnea during periods of mouth breathing or nasal blockage are also known.

The Nasal Oral Cannula of the present invention is designed to optimize patient comfort, and at the same time, produce robust nasal and oral signals. The greatest challenge in this design (and the most unique aspect) is the capture of the oral flow signal, particularly the inspiratory oral flow signal.

It is well documented that all of the important information relating to the upper airway obstruction lies in the inspiratory flow signal. Current art makes use of large scoops and baffles that hang in front of the mouth in order to capture the oral signal (see U.S. Pat. No. 6,155,986). These designs reduce patient comfort due to their bulk and are only marginally effective.

The prior art of dipping a part in a plastisol to create a coating thereof is exemplified by U.S. Pat. Nos. 3,906,071, 4,695,241, 4,800,116 and U.S. pending application Ser. No. 09/754,471 filed Jan. 4, 2001, the disclosures of which are hereby incorporated by reference.

### SUMMARY OF THE INVENTION

According to the invention there is provided a cannula, defining an interior cavity interconnecting an elongate mouthpiece, an elongate main body and at least one nare.

### BRIEF DESCRIPTION OF THE DRAWINGS

The invention will now be described, by way of example, with reference to the accompanying drawings, in which:

FIG. 1 is an orthogonal view of a cannula according to the invention;

FIG. 2 is a general cross-section of a cannula made by the method of the present invention taken in the direction of section 2-2 of FIG. 1;

FIG. 3 is a side elevation of the mouthpiece of FIGS. 1 and 2 taken on section 3-3 of FIG. 1;

FIG. 4 illustrates inspiration airflow between the lips of a patient;

FIG. 5 illustrates the placement of mouthpiece (oral prong) of the present invention relative to the lips;

FIG. 6 illustrates the location of the preferred termination of the mouthpiece between the lips of the patient;

FIG. 7 illustrates the optimal cutting angles of the end of the mouthpiece to achieve a venturi tube action during inspiration;

FIG. 8 is a block diagram of a signal conditioning circuit, for the nasal/oral cannula of the present invention; and

FIG. 9 is a comparative graphical representation of the operation of the present invention relative to a prior art device (with and without a filter).

### DETAILED DESCRIPTION OF THE EMBODIMENTS

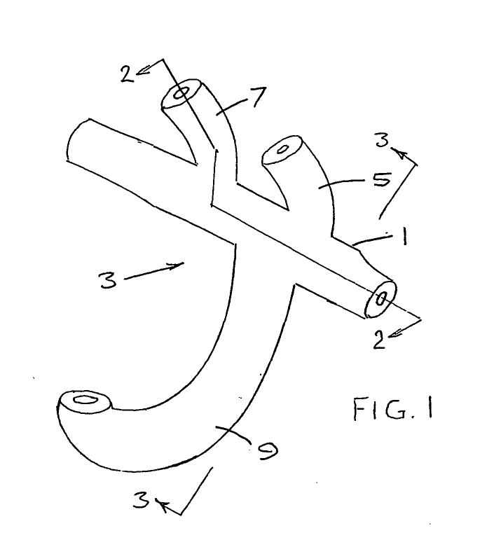

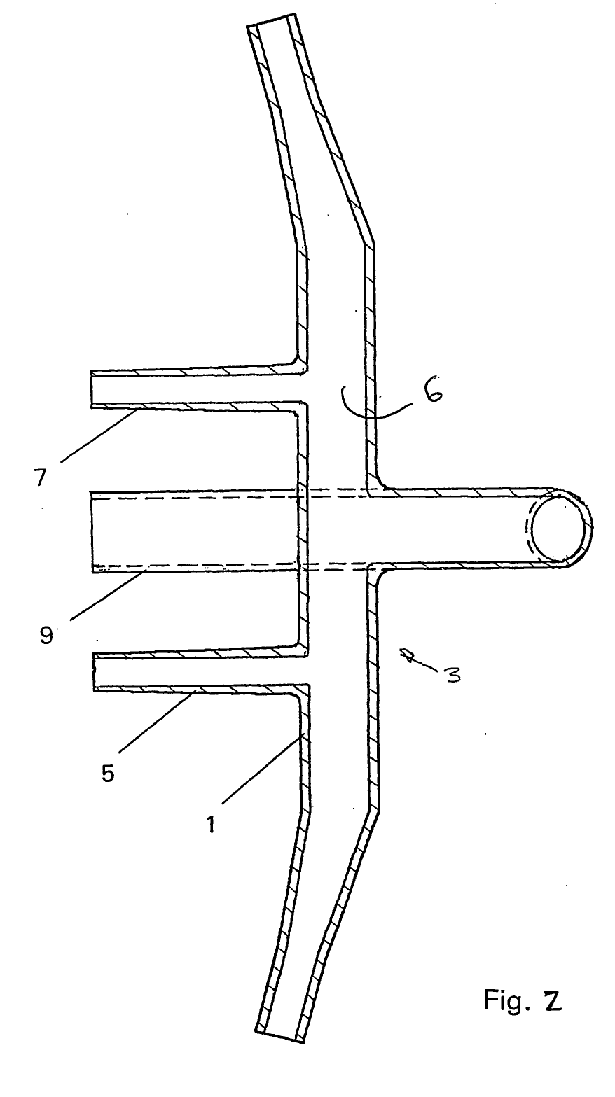

Referring to FIGS. 1 and 2, a cannula 3 comprises a main body 1, having an internal passage 6, which supports a pair of nares 5, 7 and a mouthpiece, oral prong, or inlet tube 9. The cannula 3 is composed of polyvinyl chloride (PVC).

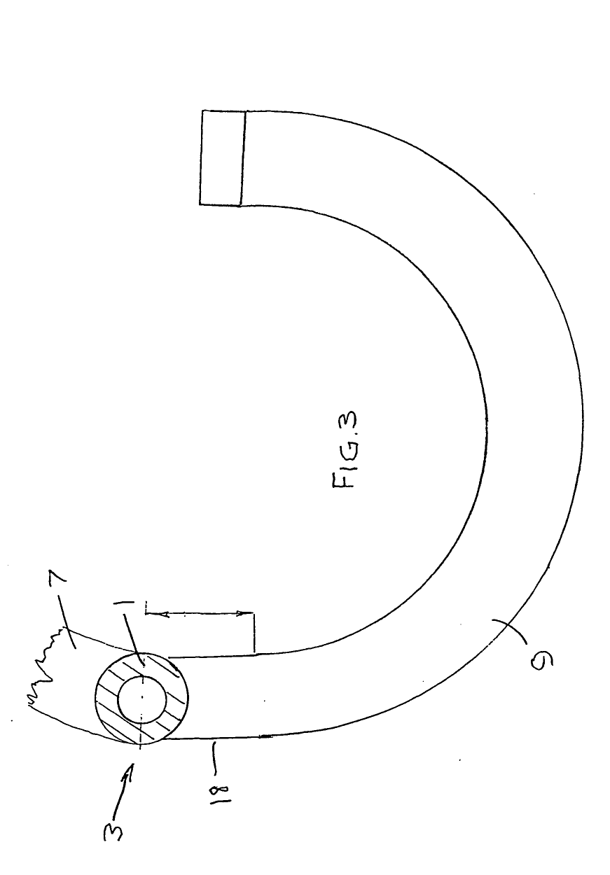

Referring now to FIG. 3 the mouthpiece 9 defines a straight portion 18 adjacent the main body of the cannula 3 which, in early prototypes, had a length from the center of the main body 1 of about 0.250 inches. It appears from later examples, however, that a shorter straight portion 18 having a length of about 0.200 inches from the center of the main body or less may provide superior performance.

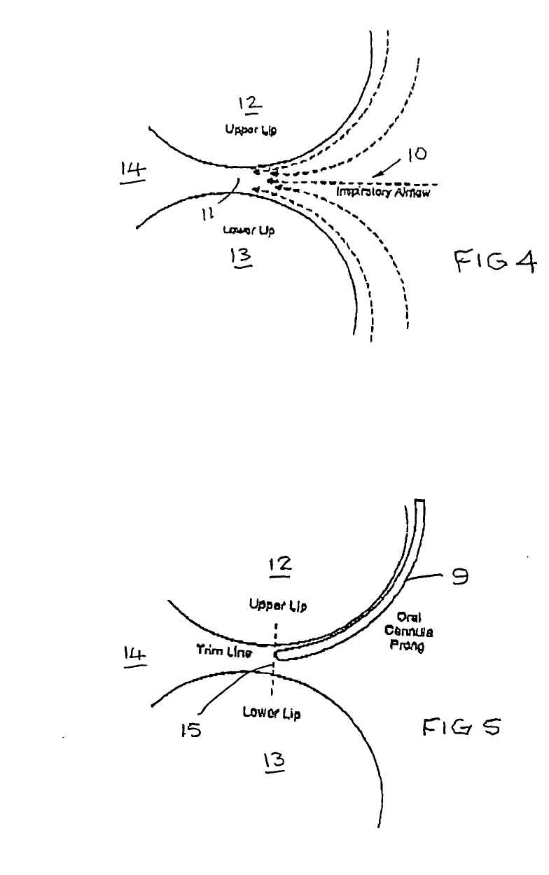

The cannula makes use of the natural funneling effect of the lips and mouth opening to produce a superior signal. FIG. 4 illustrates the inspiratory airflow 10. The surrounding air is funneled into the oral opening 11 between the lips 12, 13 and into the upper airway. The velocity of the airflow is somewhat higher over the surface of the lips than out in the middle of the oral opening. When the oral opening is large, the airflow velocity is reduced. When the oral opening is small, the airflow velocity is increased. The scoops and baffles of existing art are positioned in the middle of the opening where the velocity is always at its lowest. The inlet or opening of the oral prong 9 is positioned along the surface of the upper lip 12, in the center of the mouth, where the velocity is greatest. This placement helps to reduce the effect of varying sizes of the oral opening.

FIG. 5 shows the placement of the oral prong 9 of the cannula. The oral prong of the cannula is trimmed along a trim line 15 where the upper and lower lips 12, 13 meet with one another. This positions the inlet opening of the oral prong 9 of the cannula at the point where the flow velocity (pressure) is greatest. If the oral prong 9 is a little long, the amplitude of the inspiratory signal is not affected but it does reduce patient comfort (because the oral prong is in the mouth). If the oral prong is trimmed too short, the signal amplitude may be reduced. FIG. 6 illustrates the trim zone 16 of the best results.

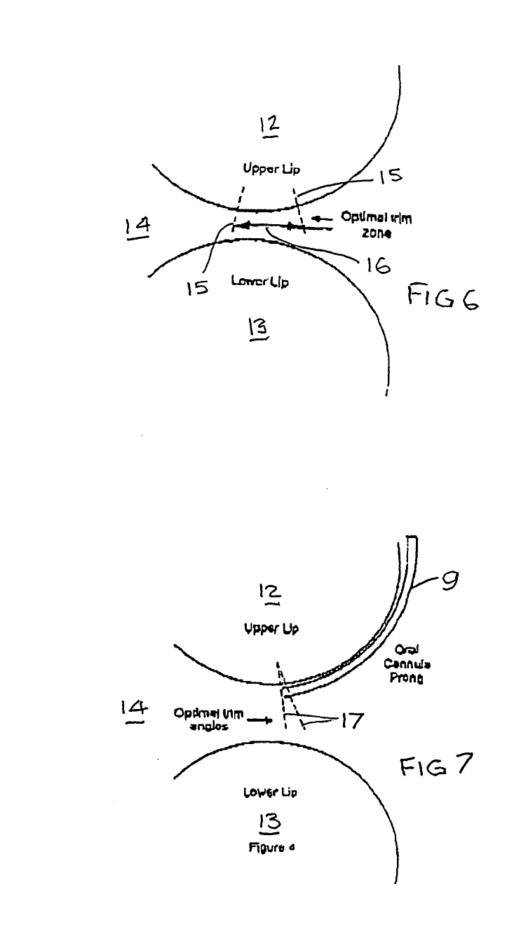

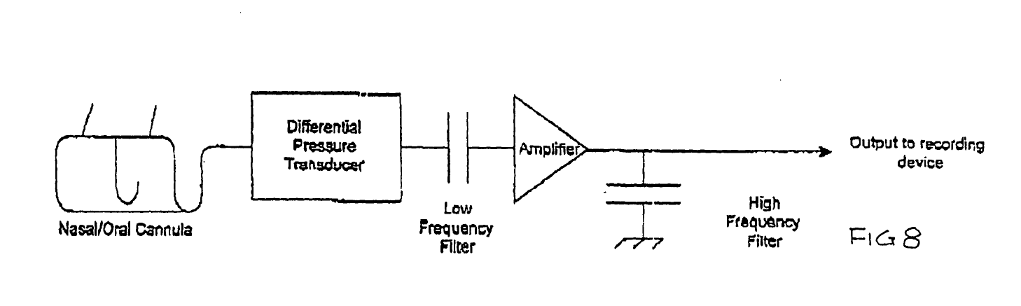

The trim angle is not particularly critical although a trim perpendicular to the direction of flow of breath through the mouth opening is preferred to optimize pressure signal acquisition. The intent of the design is to act as a venturi tube during inspiration and pitot tube during expiration. FIG. 7 illustrates the optimal cutting angle range to achieve this. When the inspiratory airflow passes over the end of the oral prong, a negative pressure is created inside of the cannula that is converted to an electrical signal by a pressure transducer and produces an upward deflection on a standard recording device. When the expiratory airflow passes over the end of the oral prong 9, a positive pressure is created inside of the cannula producing a downward deflection on the recording device. It is possible, if the trim length or trim angle is too extreme, to have a venturi tube on both inspiration and expiration which produces two upward deflections on the recording device.

With reasonable care, a properly trimmed cannula will produce a robust nasal and oral signal without compromising patient comfort with unnecessary scoops and baffles.

FIG. 8 illustrates the blocks necessary to condition the signal produced by the nasal/oral cannula. The nasal/oral cannula is connected to a sensitive pressure transducer. The range of this transducer is +/−10 inches of water. This transducer is excited by a constant voltage or constant current source. The signal produced by the transducer is then coupled to an amplifier by way of a low frequency filter with a time constant of longer than three seconds. The low frequency filter eliminates DC baseline offset while allowing for a good low frequency respiratory airflow signal. This signal is then amplified to a level useful for the recording device (e.g. +/−2 volts peak-to-peak). The output is frequency limited to 100 Hz by a high frequency filter. This eliminates any spurious signals produced by electrical interference while allowing enough high frequency response to detect a snoring signal.

To ascertain if a cannula of the present invention with an oral port can be used as a pressure sensor compare to a Breabon Cannula, the following equipment was used:

Data Instruments Differential Pressure Transducer

(DCXL01DN/8C6-1) 0.192 in H2O/V

Regain=161

[symbol]

Gain=1247.236

Vsup=5 V

Labview Data Acquisition

The inventive cannula was attached to high side of the pressure transducer.

The patient was allowed to breath through the cannula.

An analog signal was viewed.

The test was repeated for the other cannula.

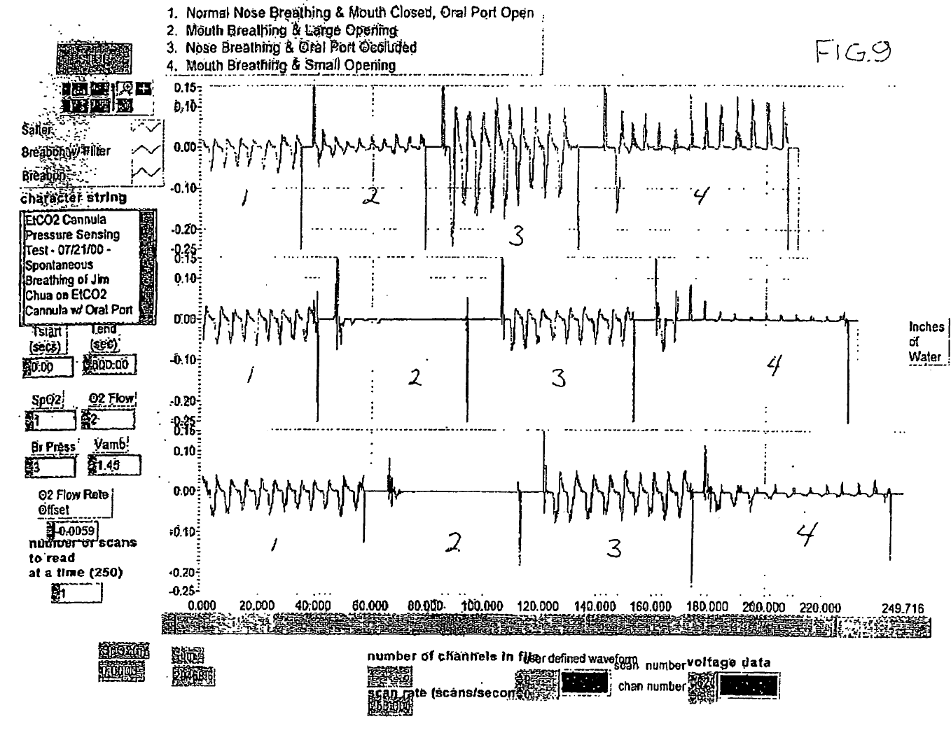

The results are shown in FIG. 9.

Conclusion: Under various breath conditions, a greater pressure signal was obtained using a Cannula of the present invention than the Breabon Cannula.

It will be further appreciated that the outer ends of the main body 1 may be trimmed to provide a discrete area where a connecting tubing may be connected thereto, e.g. by solvent bonding, and that the mouthpiece may be trimmed to a length suited to an individual patient so as to maximize the sensitivity of the finished device.

The present invention also includes a method of using the cannula of the present invention wherein the nares are placed in the nostrils of a patient and the mouthpiece is oriented and trimmed whereby the inlet or open end of the mouthpiece is located between the patients lips with the open end trimmed to extend approximately perpendicular to the direction of that air flow.

Reference Numerals
1 main body
3 cannula
5 nare mandrel
7 nare
9 mouthpiece (oral prong)
10 inspiration airflow
11 oral opening
12 upper lip
13 lower lip
14 upper airway
15 trim line
16 trim zone
17 trim angle
18 straight position

1. An improved nasal and oral cannula to facilitate detection of apnea, the cannula comprising:
a main body supporting a pair of nares, and each one of the pair of nares communicating with an internal passage of the main body to facilitate communication with a nostril of a user;
an oral prong connected to and communicating with the internal passage of the main body, the oral prong extending away from the pair of nares to facilitate insertion of a remote end, having an inlet, of the oral prong adjacent an open between a pair of lips of the user, and the remote end of the oral prong being trimmable to a shortened length so that the oral prong is positioned in the open between a pair of lips of the user, during use, to facilitate receiving a signal from the user indicative of breathing.

2. A method of fitting a nasal and oral cannula to a patient for detection of apnea, the method comprising the steps of:
placing at least one nare of a cannula in a nostril of a user;
positioning an oral prong of the cannula between lips of the user;
trimming a remote end of the oral prong of the cannula to a shortened length so that the remote end is positioned between the lips of a user; and
receiving a signal from the user via the cannula, during breathing of the user, to indicate breathing of the user.
